(19) United States
(12) Patent Application Publication
(10) Pub. No.: US 2011/0060191 A1
(43) Pub. Date: Mar. 10, 2011

(54) Musical otoscope

(76) Inventors: Jason D. Lynn, Zanesville, OH (US); Melissa A. Lynn, Zanesville, OH (US)

(21) Appl. No.: 12/807,430

(22) Filed: Sep. 3, 2010

Related U.S. Application Data
(60) Provisional application No. 61/240,008, filed on Sep. 4, 2009.

(51) Int. Cl. A61B 1/227

(52) U.S. Cl. 600/200

(57) ABSTRACT

The present invention relates to otoscopes and more specifically to musical or sound emitting otoscopes that may serve to calm pediatric patient or patients with emotional or developmental disabilities. The musical otoscope incorporates a head capable of receiving a speculum, a speaker mounted within the head and a sound reproduction apparatus for driving the speaker. The head of the otoscope may include a housing having a front side, a rear side and an interior space, the rear side having a magnifying lens, the front side receives a base of the and a speaker mounted within the housing, where the speaker is linked to a sound reproduction apparatus for driving the speaker is presented. The sound reproduction device may be an MP3 player or any other device that includes a power source, a micro controller/processor, a digital signal processor and a digital to analog converter. The use of a sound emitting otoscope has been found to ease patient diagnosis and care.

### CLAIM OF PRIORITY

Applicant hereby claims priority to U.S. Provisional Patent Application 61/240,008 which was filed on Sep. 4, 2009.

### BACKGROUND

The present invention pertains to an otoscope. More particularly, the invention pertains to an otoscope system that provides a sound or music to calm the patient.

Otoscopes are used for examination of auditory canals. A standard otoscope includes a head, a shaft and a handle. The head is secured to a shaft that extends from the handle. Generally, the handle and shaft are made of stainless steel, while the head is formed stainless steel or a hard plastic. The head of an otoscope generally includes a light source and a magnifying lens. The head typically includes a port which is connected to a conical tip or speculum, which is inserted into the auditory canal and directs the field of view. The tip of the otoscope is inserted into the auditory canals of a patient so that a medical professional may observe the condition of the canal and diagnose illness or injury and determine the best course of action for treatment.

Otoscopes are typically direct illumination scopes such as that shown in U.S. Pat. No. 3,934,578 herein incorporated by reference or reflection or refraction type scopes such as shown in U.S. Pat. No. 4,366,811 herein incorporated by reference. Recently, otoscopes such as the Welch Allyn Pocket Scope, models 228XX have been produced with an integrated handle and shaft. These integrated handles and shafts provide improved ergonomics based on improved light sources and batteries but have identical functionality to traditional otoscopes.

Children with various emotional, physical or developmental issues may be hypersensitive to touch as well as to sound. Often, an indication of autism is extreme sensitivity to touching of the ears. This sensitivity is often demonstrated in a pediatrician's office when a medical professional attempts to insert the tip of an otoscope into the auditory canal. The result can vary from pulling away from the otoscope making a diagnosis difficult to a longer emotional outburst. Repeated instances may cause a patient to form a negative association with going to the doctor office. Patients with emotional, physical or developmental issues often require healthcare well beyond that required by typical pediatric patients and a strong aversion to a doctor visit can become a long term issue in obtaining proper healthcare.

### SUMMARY

The present invention relates to otoscopes and more specifically to musical or sound emitting otoscopes that may serve to calm pediatric patient or patients with emotional or developmental disabilities.

In accordance with embodiments of the present invention, a musical otoscope incorporating a head capable of receiving a speculum, a speaker mounted within the head and a sound reproduction apparatus for driving the speaker is presented.

In accordance with additional embodiments of the present invention, an otoscope having a head with a housing, said housing having a front side, a rear side and an interior space defined therebetween, the rear side having a magnifying lens mounted therein, the front side being capable of receiving a base of a speculum, the speculum having a internal passage in visual connection with the magnifying lens and a speaker mounted within the interior space and a sound reproduction apparatus for driving the speaker is presented.

In accordance with additional embodiments of the present invention, an otoscope incorporating a head having a housing, the housing incorporating a front side capable of receiving a base of a speculum, a rear side, having a magnifying lens mounted therein and an interior space defined therebetween and having a speaker mounted therein; as well as a power source; a micro controller/processor; a digital signal processor; and a digital to analog converter linked to said speaker and driving the speaker is presented.

### BRIEF DESCRIPTION OF THE DRAWINGS

A more complete appreciation of the invention and the many embodiments thereof will be readily obtained as the same becomes better understood by reference to the following detailed description when considered in connection with the accompanying drawings, wherein:

FIG. 1 illustrates a plan view of the musical otoscope of the present invention;

FIG. 2 illustrates a plan view of a control panel suitable for use with the otoscope of the present invention;

FIG. 3 illustrates a block schematic diagram of a portable audio player suitable for use with the otoscope of the present invention.

### DETAILED DESCRIPTION

The present invention will now be described with occasional reference to the specific embodiments of the invention. This invention may, however, be embodied in different forms and should not be construed as limited to the embodiments set forth herein. Rather, these embodiments are provided so that this disclosure will be thorough and complete, and will fully convey the scope of the invention to those skilled in the art.

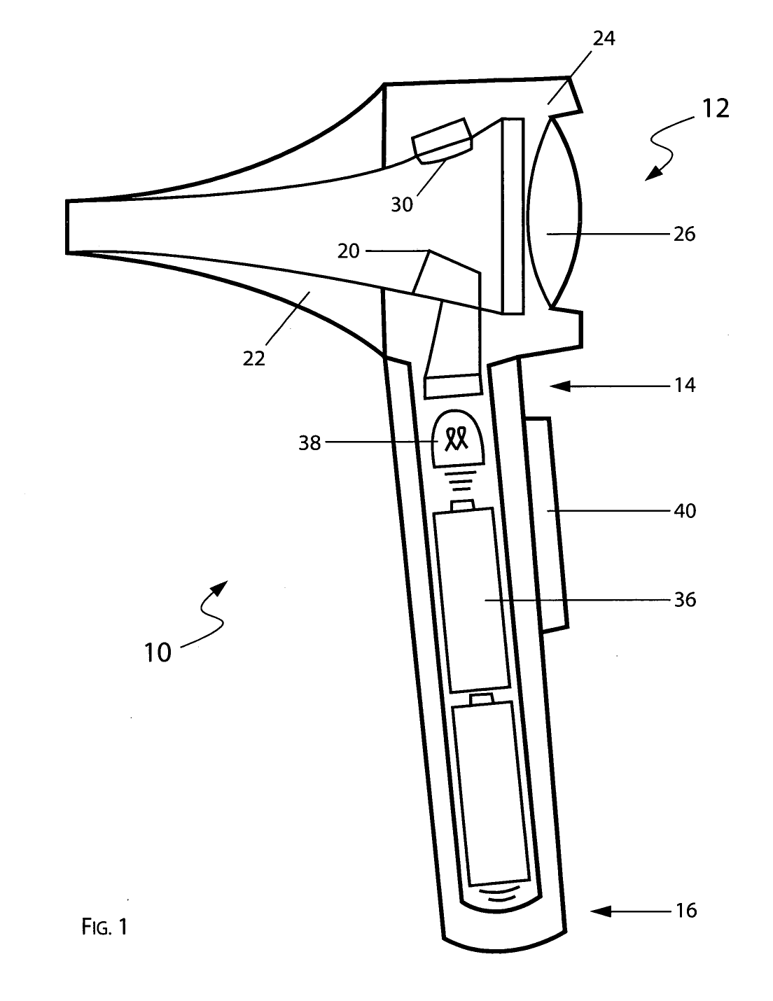

FIG. 1 is a plan view of the musical otoscope of the present invention. The otoscope 10 may include head 12, shaft 14 and handle 16. The head 12 typically includes a housing with an illuminator 10 which may be a light source of a refractive or reflective element that guides light into a speculum 22 mounted on the front of housing 24. A magnifying lens 26 may also be attached to the rear of housing 24. A speaker is provided within the head 12, preferably the speaker is mounted within the housing 24 so that speculum 22 may be replaced without interfering with the operation of the speaker. Generally, the shaft 14 and handle 16 are made of stainless steel, while the head 24 is formed stainless steel or polymer. The handle 16 typically includes an internal power source 36 (a battery is shown however the otoscope may be connected to a wall mounted power source (not shown), a light source 38 and a control panel 40.

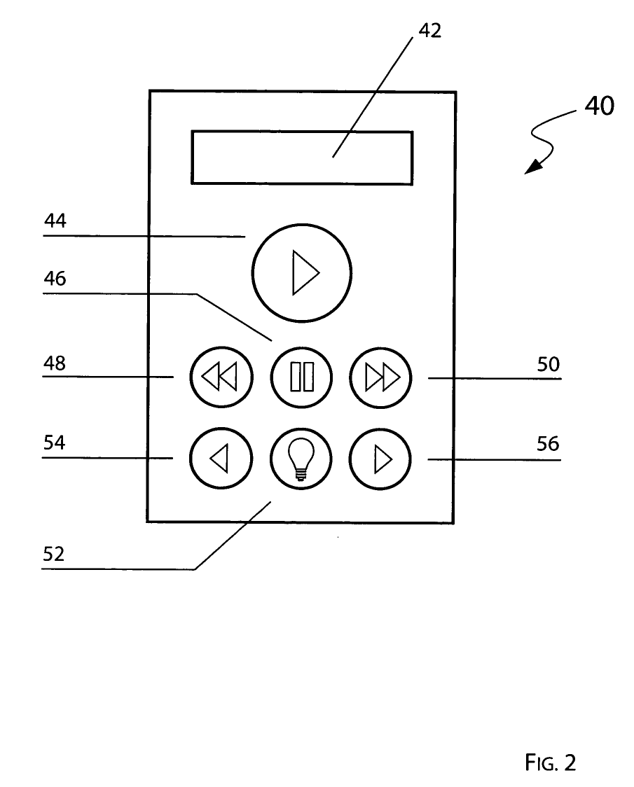

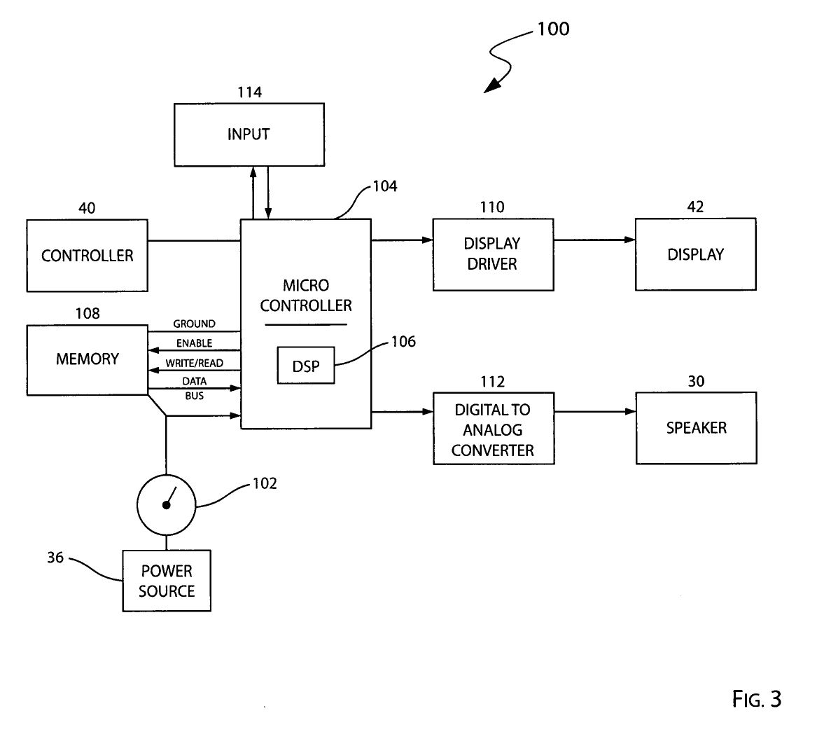

FIG. 2 is a plan view of the control panel 40 of the otoscope of the present invention. The control panel 40 may include status display 42, play button 44, pause button 46, and program selection buttons 48, 50. The structure of the audio player may be housed within the control panel 40. The audio player may be a basic audio player such as an MP3 player as shown in FIG. 3. Play lists may be preloaded based on age and gender and individual play lists may be selected using buttons 48, 50. The control panel 40 may also include a switch 52 for powering on the light source 38 as well as light control switches 54, 56 for controlling the brightness of the light source. Play lists may be preloaded with the otoscope based on age, gender and interests of a patient or may be customized by end user.

FIG. 3 is a block schematic diagram of the audio player 100. The player may be contained within the control panel 40 of otoscope 10 or may be integrated with the handle 16, neck 14 or head 12. The player 100 may include a power source 36 such as the rechargeable batteries used to illuminate the otoscope 12 or a separate power source (not shown) connected to player 100 by on/off switch 102. Player 100 typically will include a micro controller/processor 104 and a digital signal processor (DSP) 106. DSP 106 and micro controller/processor 104 are linked by a bus. Micro controller/processor 104 may receive signals from control buttons 44, 46, 48, 50 and manages information flow to and from memory 108 and to status display 42. Micro controller/processor 104 may be linked to display driver 110 for controlling the output signal shown on display 42. Micro controller/processor 104 may be linked to a digital to analog converter 112 that drives speaker 30 to output pleasing sounds or songs to help comfort or calm a patient.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. Any numerical values, however, inherently contain certain errors necessarily resulting from error found in their respective measurements.

The present invention should not be considered limited to the specific examples described herein, but rather should be understood to cover all aspects of the invention. Various modifications, equivalent processes, as well as numerous structures and devices to which the present invention may be applicable will be readily apparent to those of skill in the art. One example would be that although the device is called a musical otoscope the sound output from the speaker in the present invention may be music, a short story, and a series of animal sounds or a pattern of calming tones. Those skilled in the art will understand that various changes may be made without departing from the scope of the invention, which is not to be considered limited to what is described in the specification.

What is claimed is:

1. A musical otoscope comprising:
a head capable of receiving a speculum;
a speaker mounted within said head; and
a sound reproduction apparatus for driving said speaker.

2. The musical otoscope of claim 1, wherein the sound reproduction apparatus further comprises:
a power source;
a micro controller/processor;
a digital signal processor; and
a digital to analog converter linked to said speaker.

3. The musical otoscope of claim 2, further comprising:
a control panel linked to said micro controller/processor.

4. The musical otoscope of claim 1, further comprising:
a light source;
a handle mounted to said head, the handle having a power source.

5. An otoscope comprising:
a head having a housing, said housing having a front side, a rear side and an interior space defined therebetween, the rear side having a magnifying lens mounted therein, the front side being capable of receiving a base of a speculum, the speculum having a internal passage in visual connection with the magnifying lens and a speaker mounted within the interior space; and
a sound reproduction apparatus for driving said speaker.

6. The otoscope of claim 5 wherein the sound reproduction apparatus includes a power source, a micro controller/processor, a digital signal processor and a digital to analog converter linked to said speaker.

7. The otoscope of claim 5, further comprising:
a handle attached to said head, the handle including a light source.

8. The otoscope of claim 7, further comprising:
a reflective element within said head for guiding light generated from said light source through said speculum.

9. The otoscope of claim 7, further comprising:
a refractive element within said head for guiding light generated from said light source through said speculum.

10. The otoscope of claim 5, further comprising:
a light source mounted within said head.

11. The otoscope of claim 5, further comprising:
a neck having first and second ends, the first end linked to and supporting said head;
a handle linked to and supporting the second end of said neck; and
a light source in optical communication with the internal passage of said head.

12. The otoscope of claim 11, further comprising:
a power source mounted within said handle.

13. An otoscope comprising:
a head having a housing, said housing having:
a front side capable of receiving a base of a speculum,
a rear side, having a magnifying lens mounted therein and
an interior space defined therebetween and having a speaker mounted therein;
a power source;
a micro controller/processor;
a digital signal processor; and
a digital to analog converter linked to said speaker and driving said speaker.

14. The otoscope of claim 13, further comprising:
a light source

15. The otoscope of claim 13, further comprising:
a handle attached to said head, the handle including a light source.
